(19) United States
(12) Patent Application Publication
(10) Pub. No.: US 2008/0261937 A1
(43) Pub. Date: Oct. 23, 2008

(54) PHARMACEUTICAL COMPOSITIONS AND METHOD FOR TREATING PEDIATRIC HYPOGONADISM

(76) Inventors: Robert E. Dudley, Rosemary Beach, FL (US); Alan Rogol, Charlottesville, VA (US); Janet Benesh, Kennesaw, GA (US); Troy L. Zumbrunnen, Roswell, GA (US); Gregg Pratt, Canton, GA (US); John J. Brennan, Roswell, GA (US); Hjalmar Lagast, Roswell, GA (US)

Correspondence Address: MAYER BROWN LLP, P.O. BOX 2828, CHICAGO, IL, 60690, US

(21) Appl. No.: 12/052,337

(22) Filed: Mar. 20, 2008

Related U.S. Application Data
(60) Provisional application No. 60/896,552, filed on Mar. 23, 2007.

(51) Int. Cl. A61K 31/568; A61P 5/00

(52) U.S. Cl. 514/178

(57) ABSTRACT

The present invention relates to compositions for treating prepubertal males of adolescent age with insufficient testosterone production using a hydroalcoholic testosterone gel formulation that provides, among other things, a desirable pharmacokinetic hormone profile, and methods of treating said adolescent males.

This application claims priority to U.S. Provisional Patent Application Ser. No. 60/896,552 filed Mar. 23, 2007, the entire contents of which is hereby incorporated by reference herein.

### BACKGROUND OF THE INVENTION

The age of onset of puberty in boys ranges from nine to fourteen years and is characterized by testicular enlargement followed by the appearance of pubic hair eighteen to twenty-four months after the onset of testicular growth. Puberty can be characterized by skeletal growth, with linear growth velocity beginning to increase at Tanner Genital Stage III and Tanner Pubic Hair Stage II. The Tanner Stages (I to V) are stages of physical development in children and adolescents. The stages define physical measurements of development based on external primary and secondary sex characteristics, such as the development of pubic hair. Due to natural variation, individuals pass through the Tanner Stages at different rates, depending in particular on the timing of puberty. Peak height velocity is typically reached at 14 years of age. Wheeler, M D, Endocrinol and Metab Clin N. Am., 20(1):1-14 (1991). Boys who do not begin secondary sexual development by age 14 years or who do not progress through Stage V pubertal development within 4.5 years after the onset of puberty should be evaluated for hypogonadism, i.e., low testosterone levels. Styne, D., Puberty, Basic and Clinical Endocrinology, 6^{th} Edition, Greenspan F S and Gardner D G, ed. McGraw-Hill, New York, 2001.

Testosterone, the major circulating androgen in males, is synthesized from cholesterol. It is primarily secreted in the testes of males. In the adult male, the approximately 500 million Leydig cells in the testes secrete more than 95% of the 6-7 mg of testosterone produced per day. Two hormones produced by the pituitary gland, luteinizing hormone (“LH”) and follicle stimulating hormone (“FSH”), are required for the development and maintenance of testicular function and negatively regulate testosterone production via a feedback mechanism driven by circulating concentrations of the hormone. Circulating testosterone is metabolized to various 17-keto steroids through two different pathways. Testosterone can be metabolized to dihydrotestosterone (“DHT”) by the enzyme 5α-reductase or to estradiol (“E2”) by an aromatase enzyme complex.

Testosterone circulates in the blood 98% bound to proteins. In males, approximately 40% of the binding is to the high-affinity sex hormone binding globulin (“SHBG”). The remaining 60% is bound weakly to albumin. Thus, a number of measurements for testosterone are available from clinical laboratories. The term “free” testosterone as used herein refers to the fraction of testosterone in the blood that is not bound to protein. The term “total testosterone” or “testosterone” as used herein means the free testosterone plus protein-bound testosterone. The term “bioavailable testosterone” as used herein refers to the non-SHBG bound testosterone and includes testosterone weakly bound to albumin.

The following table summarizes the normal testosterone concentration ranges for each Tanner Stage:

TABLE 1
Testosterone Levels in Males by Tanner Stage

| Tanner Stage | Normal Range | |
|---|---|---|
| I (prepubertal stage) | 2 to 23 | ng/dL |
| II | 5 to 70 | ng/dL |
| III | 15 to 280 | ng/dL |
| IV | 105 to 545 | ng/dL |
| V | 265 to 800 | ng/dL |

DeGroot, Leslie, Endocrinology, 4^{th} Edition, W. B. Saunders Company, New York, 2001.

The following table summarizes the serum hormone concentration ranges in normal males by age group:

TABLE 2
Hormone Levels in Adolescent Males by Age Group

| Age | Hormone | Normal Range | |
|---|---|---|---|
| 10-11 Years | Total Testosterone | 5 to 50 | ng/dL |
| | Free Testosterone | 0.6 to 5.7 | ng/dL |
| 12-14 Years | Total Testosterone | 10 to 570 | ng/dL |
| | Free Testosterone | 1.4 to 156 | ng/dL |
| 15-17 Years | Total Testosterone | 220 to 800 | ng/dL |
| | Free Testosterone | 80 to 159 | ng/dL |

DeGroot, Leslie, Endocrinology, 4^{th} Edition, W. B. Saunders Company, New York, 2001.

There is considerable variation in the half-life of testosterone reported in the literature, ranging from 10 to 100 minutes. Researchers do agree, however, that circulating testosterone has a diurnal variation in normal young men. Maximum levels occur at approximately 6:00 to 8:00 a.m. with levels declining throughout the day.

Delayed puberty in adolescent males (i.e., boys) may result from different conditions. For example, it may result from Constitutional Delay in Growth and Puberty (CDGP), hypergonadotropic hypogonadism (primary hypogonadism), or hypogonadotropic hypogonadism (secondary hypogonadism). For testosterone naïve subjects, prepubertal maturation status can be indicated by, among other things: (i) testis volume of ≦3 mL and (ii) testosterone concentration of ≦50 ng/dL.

Hypogonadism results from a variety of patho-physiological conditions in which testosterone concentration is diminished below the normal range. The hypogonadic condition is sometimes linked with a number of physiological changes, such as reduced lean body mass, decreased bone density, lowered mood, and decreased energy levels.

Researchers generally classify hypogonadism into one of three types. Primary hypogonadism includes the testicular failure due to congenital or acquired anorchia, XYY Syndrome, XX males, Noonan's Syndrome, gonadal dysgenesis, Leydig cell tumors, maldescended testes, varicocele, Sertoli-Cell-Only Syndrome, cryptorchidism, bilateral torsion, vanishing testis syndrome, Klinefelter's Syndrome, chemotherapy, toxic damage from alcohol or heavy metals, and general disease (renal failure, liver cirrhosis, diabetes, myotonia dystrophica). Patients with primary hypogonadism show an intact feedback mechanism in that the low serum testosterone concentrations are associated with high FSH and LH concentrations. However, because of testicular or other failures, the high LH concentrations are not effective at stimulating testosterone production.

Secondary hypogonadism involves an idiopathic gonadotropin or LH-releasing hormone deficiency. This type of hypogonadism includes Kallman's Syndrome, Prader-Labhart-Willi's Syndrome, Laurence-Moon-Biedl's Syndrome, pituitary insufficiency/adenomas, Pasqualini's Syndrome, hemochromatosis, hyperprolactinemia, or pituitary-hypothalamic injury from tumors, trauma, radiation, or obesity. Because patients with secondary hypogonadism do not demonstrate an intact feedback pathway, the lower testosterone concentrations are not associated with increased LH or FSH levels. Thus, these males have low testosterone serum levels but have gonadotropins in the normal to low range.

Adolescent males with delayed puberty associated with the conditions described above may be treated with androgens (e.g., testosterone) or anabolic steroids. Adolescent males with permanent hypogonadism will require long-term androgen supplementation. Such treatment will typically produce secondary sexual development and an increase in stature. The most common form of testosterone used for treatment of delayed puberty is the injectable form. This is a depot formulation in which a testosterone ester (e.g., testosterone enanthate) is dissolved in oil and injected deeply into the gluteal muscle every few weeks. This regimen requires frequent visits to the physician's office and is painful. Injections of testosterone also result in serum testosterone concentrations that fluctuate widely over the dosing interval, from higher than desired immediately after an injection to lower than desired before the next injection. These fluctuating concentrations over the dosing interval complicate the use of serum testosterone concentrations as a meaningful indicator for dosage adjustments. Oral halogenated or methylated testosterone products are not popular in the United States because of the risk of hepatic complications. Furthermore, use of the anabolic steroids does not promote increased secretion of growth hormone, as does testosterone. Thus, disadvantages are associated with each of the products typically used to treat delayed puberty.

In 2000, a testosterone gel 1% was approved for replacement therapy in adult males over 18 years of age for conditions associated with a deficiency or absence of endogenous testosterone (primary and secondary hypogonadism). Results of a testosterone-replacement study in 73 hypogonadal men using 5 g of testosterone gel 1% (containing 50 mg of testosterone) once daily and 78 hypogonadal men using 10 g of testosterone gel 1% (containing 100 mg of testosterone) once daily showed that testosterone gel 1% was well-tolerated and was effective in increasing serum testosterone concentrations to within eugonadal ranges. Eugonadal concentrations were achieved within a few hours of the first application in the majority of the men, and these concentrations were maintained for up to 180 days with once daily dosing. The most frequently reported adverse events (AEs) related to the use of testosterone gel 1% were acne (up to 8%); clinical laboratory test abnormalities (up to 6%) that included increased red blood cells, hemoglobin, hematocrit, and decreased serum lipids; application site reactions (up to 5%); prostate disorder (up to 5%); and headache (up to 4%).

Although there has been evidence that at doses of over 5 g or greater of testosterone gel, the serum testosterone concentrations of hypogonadal adult men over 18 years of age were increased to within eugonadal ranges, there is no corresponding evidence in the prior art that at lower dose levels the serum testosterone concentrations of hypogonadal adolescent males under 18 years of age will increase to within eugonadal ranges, nor can those dose levels be predicted. Specifically, the manner in which the skin of adolescent males will absorb testosterone gel is not clear. In general, the skin of adolescent males, when acting as a reservoir, is different from that of adult males over 18 years of age. Consequently, it is not evident that at lower dose levels of testosterone gel the blood serum testosterone concentrations of hypogonadal adolescent males will increase in a safe and effective manner. Furthermore, the uptake of testosterone from a skin reservoir depends upon the metabolism of the individual. The metabolism of adolescent males is dramatically different from that of adult males over 18 years of age.

Testosterone gel could provide several advantages for the treatment of delayed puberty in boys of adolescent age. Most importantly, the relatively consistent serum testosterone concentrations achieved with this product would allow a clinician to obtain meaningful measurements of serum testosterone concentrations to adjust the dose to attain a testosterone concentration appropriate for a given stage of pubertal development. Testosterone concentrations gradually increase as boys move from Tanner Pubic Hair Stage I through Tanner Pubic Hair Stage V. The ability to attain consistent testosterone concentrations over time and to use those concentrations to make appropriate adjustments in dose should allow clinicians to induce secondary sexual development and to move these boys through the various stages of puberty in a more physiologic manner.

An additional consideration in the use of testosterone gel for the treatment of delayed puberty in adolescent boys is convenience of use. Use of the gel would not require that the boys return to the physician's office every two to four weeks, as they do for injections. This is an important factor for both the boys and their families. Finally, the gel should be well-tolerated in this population. Its use will avoid the pain and discomfort associated with the testosterone injections and foster compliance with a testosterone therapy treatment plan. Neither will there be the risk of hepatic complications associated with the use of oral anabolic agents. The gel has been very well tolerated in the adult population, and few subjects experience application site reactions.

Accordingly, there is a need in the art for a safe and effective treatment for treating pediatric hypogonadism, i.e., low testosterone levels in adolescent males aged nine (9) to seventeen (17) years of age.

### SUMMARY OF THE INVENTION

The present invention relates to compositions for treating prepubertal males of adolescent age with insufficient testosterone production using a hydroalcoholic testosterone gel formulation that provides, among other things, a desirable pharmacokinetic hormone profile, and methods of treating said adolescent males.

### BRIEF DESCRIPTION OF THE DRAWINGS

FIG. 1 is a graph showing the mean observed serum concentration versus time profile for total and free testosterone, dihydrotestosterone, and bioavailable testosterone.

FIG. 2 is a graph showing the mean baseline-adjusted serum concentration versus time profile for total and free testosterone, dihydrotestosterone, and bioavailable testosterone.

FIG. 3 is a graph showing the observed and baseline-adjusted mean predose testosterone concentrations.

### DETAILED DESCRIPTION OF THE INVENTION

While the present invention may be embodied in many different forms, several specific embodiments are discussed herein with the understanding that the present disclosure is to be considered only as an exemplification of the principles of the invention, and it is not intended to limit the invention to the embodiments illustrated.

The present invention relates to compositions for treating prepubertal males of adolescent age, i.e., between 9 and 17 years of age (inclusive), with insufficient testosterone production (i.e., pediatric hypogonadism) using a hydroalcoholic testosterone gel formulation that provides, among other things, a desirable pharmacokinetic hormone profile, and methods using such compositions for such treatment.

For testosterone naïve subjects, prepubertal maturation status is indicated by, among other things: (i) testis volume of ≦3 mL and (ii) testosterone concentration of ≦50 ng/dL.

In one embodiment, the present invention is directed to a method for percutaneous administration of testosterone in a hydroalcoholic gel. The gel comprises testosterone (or a testosterone derivative), one or more lower alcohols, such as ethanol or isopropanol; a penetration enhancing agent such as isopropyl myristate; a thickener; and water. Additionally, the present invention may optionally include salts, emollients, stabilizers, antimicrobials, fragrances, and propellants.

The present invention also includes kits, methods, combinations, and pharmaceutical compositions for treating, preventing, reversing, halting or slowing the progression of hypogonadism or other low-testosterone-associated disorders in a subject once it becomes clinically evident, or treating the symptoms associated with, or related to the hypogonadism or low-testosterone-associated disorder. The subject may already have a diagnosis of hypogonadism and/or low testosterone at the time of administration, or be at risk of developing hypogonadism and/or low testosterone. The present invention preferably is for treatment of adolescent subjects under 18 years of age. Even more preferably, the present invention is for treatment of prepubertal subjects between 9 and 17 years of age (inclusive).

The term “derivative” refers to a compound that is produced from another compound of similar structure by the replacement of substitution of one atom, molecule or group by another. For example, a hydrogen atom of a compound may be substituted by alkyl, acyl, amino, etc., to produce a derivative of that compound.

As used herein, the term “lower alcohol,” alone or in combination, means a straight-chain or branched-chain alcohol moiety containing one to about six carbon atoms. In one embodiment, the lower alcohol contains one to about 4 carbon atoms, and in another embodiment the lower alcohol contains two to about 3 carbon atoms. Examples of such alcohol moieties include methanol, ethanol, ethanol USP (i.e., 95% v/v), n-propanol, isopropanol, n-butanol, isobutanol, sec-butanol, and tert-butanol.

As used herein, the term “lower alkyl”, alone or in combination, means a straight-chain or branched-chain alkyl radical containing one to about six carbon atoms. In one embodiment, the lower alkyl contains one to about four carbon atoms. Examples of such radicals include methyl, ethyl, n-propyl, isopropyl, n-butyl, isobutyl, sec-butyl, and tert-butyl.

As used herein, the term “ethanol” refers to C₂H₅OH. It may be used as dehydrated alcohol USP, alcohol USP, or in any common form including in combination with various amounts of water.

The composition is used in a “pharmacologically effective amount.” This means that the concentration of the drug administered is such that in the composition it results in a therapeutic level of drug delivered over the term that the drug is to be used. Such delivery is dependent on a number of variables including the time period for which the individual dosage unit is to be used, the flux rate of the drug from the composition, for example, testosterone, from the gel, surface area of application site, etc. For testosterone, for example, the amount of testosterone necessary can be experimentally determined based on the flux rate of testosterone through the gel, and through the skin when used with and without enhancers.

The term “prodrug” refers to a drug or compound in which the pharmacological action (active curative agent) results from conversion by metabolic processes within the body. Prodrugs are generally considered drug precursors that, following administration to a subject and subsequent absorption, are converted to an active or a more active species via some process, such as a metabolic process. Other products from the conversion process are easily disposed of by the body. Prodrugs generally have a chemical group present on the prodrug which renders it less active and/or confers solubility or some other property to the drug. Once the chemical group has been cleaved from the prodrug the more active drug is generated. Prodrugs may be designed as reversible drug derivatives and utilized as modifiers to enhance drug transport to site-specific tissues. The design of prodrugs to date has been to increase the effective water solubility of the therapeutic compound for targeting to regions where water is the principal solvent. For example, Fedorak, et al., Am. J. Physiol, 269:G210-218 (1995), describe dexamethasone-beta-D-glucuronide. McLoed, et al., Gastroenterol., 106:405-413 (1994), describe dexamethasone-succinate-dextrans. Hochhaus, et al., Biomed. Chrom., 6:283-286 (1992), describe dexamethasone-21-sulphobenzoate sodium and dexamethasone-21-isonicotinate. Additionally, J. Larsen and H. Bundgaard [Int. J. Pharmaceutics, 37, 87 (1987)] describe the evaluation of N-acylsulfonamides as potential prodrug derivatives. J. Larsen et al., [Int. J. Pharmaceutics, 47, 103 (1988)] describe the evaluation of N-methylsulfonamides as potential prodrug derivatives. Prodrugs are also described in, for example, Sinkula et al., J. Pharm. Sci., 64:181-210 (1975). Other nonlimiting examples of “prodrugs” that can be used in the combinations and methods of the present invention include parecoxib (propanamide, N-[[4-(5-methyl-3-phenyl-4-isoxazolyl)phenyl]sulfonyl]-), and MAG-camptothecin.

In one embodiment, the present invention is directed to a method for percutaneous administration of testosterone in a hydroalcoholic gel. The gel comprises one or more lower alcohols, such as ethanol or isopropanol; a penetration enhancing agent; a thickener; and water. In one embodiment, the gel comprises an anionic polymer thickening agent precursor neutralized, preferably neutralized with a hydroxide releasing agent, such as sodium hydroxide. Additionally, the present invention may optionally include salts, emollients, stabilizers, antimicrobials, fragrances, and propellants.

Included in the methods and pharmaceutical compositions of the present invention are the isomeric forms and tautomers of the described compounds and the pharmaceutically-acceptable salts thereof. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, b-hydroxybutyric, galactaric and galacturonic acids.

Non-limiting examples of penetration enhancing agents include C8-C22 fatty acids such as isostearic acid, octanoic acid, and oleic acid; C8-C22 fatty alcohols such as oleyl alcohol and lauryl alcohol; lower alkyl esters of C8-C22 fatty acids such as ethyl oleate, isopropyl myristate, butyl stearate, and methyl laurate; di(lower)alkyl esters of C6-C22 diacids such as diisopropyl adipate; monoglycerides of C8-C22 fatty acids such as glyceryl monolaurate; tetrahydrofurfuryl alcohol polyethylene glycol ether; polyethylene glycol, propylene glycol; 2-(2-ethoxyethoxy)ethanol; diethylene glycol monomethyl ether; alkylaryl ethers of polyethylene oxide; polyethylene oxide monomethyl ethers; polyethylene oxide dimethyl ethers; dimethyl sulfoxide; glycerol; ethyl acetate; acetoacetic ester; N-alkylpyrrolidone; and terpenes.

The thickening agents (aka gelling agents) suitable for use in the present invention include neutralized anionic polymers such as polyacrylic acid. Preferred are the carbomer polyacrylic acids, especially those made and sold by Noveon Inc. of Cleveland, Ohio under the trademark Carbopol®. (See information at http://www.noveon.com, incorporated herein by reference.) Particularly preferred are Carbopols® Ultrez 10, 940, 941, 954, 980, 981, ETD 2001, EZ-2 and EZ-3. Most preferred are Carbopol® 940 and Carbopol® 980. Other suitable anionic polymers include carboxypolymethylene and carboxymethyl cellulose. Also suitable are other known polymeric thickening agents such as Pemulen® polymeric emulsifiers, and Noveon® polycarbophils. Additional thickening agents, enhancers and adjuvants may generally be found in Remington's The Science and Practice of Pharmacy, Meade Publishing Co., United States Pharmacopeia/National Formulary, all incorporated herein by reference.

In one embodiment, the formulation contains an anionic polymer thickening agent precursor such as a carbomer which has been combined with a neutralizer selected from the group consisting of sodium hydroxide, ammonium hydroxide, potassium hydroxide, arginine, aminomethyl propanol, tetrahydroxypropyl ethylenediamine, triethanolamine (“TEA”), tromethamine, PEG-15 cocamine, diisopropanolamine, and triisopropanolamine, or combinations thereof in an amount sufficient to neutralize the anionic polymer thickening agent precursor to form a gel in the course of forming the composition. Suitable neutralizing agents and their use with selected anionic polymer thickening agent precursors are disclosed in “Neutralizing Carbopol® and Pemulen® Polymers in Aqueous and Hydroalcoholic Systems,” Commercial Brochure TDS-237 (October 1998) by Noveon Inc. of Cleveland, Ohio, incorporated by reference herein.

In another embodiment, the formulation of the present invention delivers about 0.5 mg to about 50 mg testosterone, or the equivalent thereof, to a subject per dosage unit. In another embodiment of the present invention, the formulation delivers from about 5 mg to about 25 mg testosterone, or the equivalent thereof, to a subject per dosage unit. In yet another embodiment of the present invention, the formulation delivers from about 5 mg to about 15 mg testosterone, or the equivalent thereof, to a subject per dosage unit. In another embodiment of the present invention, the formulation delivers from about 15 mg to about 25 mg testosterone, or the equivalent thereof, to a subject per dosage unit. In still another embodiment of the present invention, the formulation delivers from about 25 mg to about 50 mg testosterone, or the equivalent thereof, to a subject per dosage unit. Thus, for example, a testosterone gel, ointment, cream or patch formulated for once a day administration can contain about 5 mg, or about 15 mg, or about 25 mg, or about 50 mg testosterone.

In one embodiment, the formulation is a gel, an ointment, a cream or a patch and is comprised of testosterone; a penetration enhancing agent, such as isopropyl myristate; a thickening agent, such as a neutralized carbomer; a lower alcohol, such as ethanol or isopropanol; and water. In another embodiment the formulation is a gel, an ointment, a cream or a patch and is comprised of the following substances in approximate percentages:

TABLE 3
Composition of Testosterone Formulation

| SUBSTANCE | AMOUNT (w/w) |
|---|---|
| Testosterone | 0.01-15% |
| Penetration | 0.01-50% |
| enhancing agent | |
| Gelling agent | 0.01-50% |
| Lower alcohol | 30-98% |
| Purified water (qs) | to 100% |

In another embodiment, the formulation contains an anionic polymer thickening agent precursor such as a carbomer which has been combined with a neutralizer in an amount sufficient to form a gel in the course of forming the composition.

In yet a further embodiment, the formulation contains an anionic polymer thickening agent precursor such as a carbomer which has been combined with a neutralizer which is an aqueous solution of sodium hydroxide such as 0.1 N sodium hydroxide, or 1.5 N sodium hydroxide, or 2.0 N sodium hydroxide or any other convenient strength aqueous solution in an amount sufficient to form a gel. In one embodiment, the composition was prepared using between about 1.0% and 10.0% 0.1 N sodium hydroxide. Accordingly, embodiments employing any percentage between about 1.0% and about 10.0% 0.1 N NaOH may be used, such as, e.g., 1.0%, 2.0%, 3.0%, 4.0%, 5.0%, 6.0%, 7.0%, 8.0%, 9.0% or 10.0% 0.1 N NaOH

In one embodiment, in a 100 g composition, the gel, ointment, cream, or patch may contain about 0.01 g to about 15 g of testosterone, about 0.01 g to about 50 g penetration enhancing agent, about 0.1 g to about 50 g gelling agent, and about 30 g to about 98 g lower alcohol. In another embodiment, in a 100 g composition, the gel, ointment, cream, or patch may contain about 0.1 g to 10 g of testosterone, about 0.1 g to about 5 g of penetration enhancing agent, about 0.1 g to about 5 g of gelling agent, and about 45 g to about 90 g lower alcohol and water.

In another embodiment, the composition comprises about 0.75% to about 1.2% (w/w) testosterone; about 0.6% to about 1.2% (w/w) isopropyl myristate; about 60% to about 80% (w/w) alcohol selected from the group consisting of ethanol and isopropanol; a sufficient amount of a thickening agent to give the composition a viscosity in excess of about 9000 cps; and water.

In an embodiment, the viscosity of the composition of the present invention is about 9,000 cps to about 29,000 cps. Accordingly, the viscosity of the composition of the present invention may be any amount between about 9,000 cps and 29,000 cps, such as, e.g., 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000 or 29,000 cps.

In one embodiment of the present invention, the composition is obtained by combining about 1.0% (w/w) testosterone; about 0.6% to about 1.4% (w/w) isopropyl myristate; about 67% to about 74% (w/w) ethanol; about 0.6% to about 1.4% (w/w) carbomer; about 6.5% to about 7.5% (w/w) 0.1N NaOH; and additional water.

In another embodiment of the present invention, the composition is obtained by combining about 0.9% to 1.1% (w/w) testosterone; about 0.4% to about 0.6% (w/w) isopropyl myristate; about 68% to about 73% (w/w) ethanol; about 0.85% to about 0.95% (w/w) carbomer; about 4.6% to about 4.9% (w/w) 0.1N NaOH; and additional water.

In various instances, it may be preferable to utilize higher testosterone concentrations. Hence, in yet another embodiment of the present invention, the composition is obtained by combining about 1.15% to 1.8% (w/w) testosterone; about 0.6% to about 1.2% (w/w) isopropyl myristate; about 60% to about 80% (w/w) ethanol; about 0.6% to about 1.4% (w/w) carbomer; and additional water. In another example, the composition may additionally contain a neutralizer which is an aqueous solution of sodium hydroxide such as 0.1 N sodium hydroxide, or 1.5 N sodium hydroxide, or 2.0 N sodium hydroxide or any other convenient strength aqueous solution in an amount sufficient to form a gel. In one embodiment, the composition was prepared using between about 1.0% and 10.0% 0.1 N sodium hydroxide. Accordingly, embodiments employing any percentage between about 1.0% and about 10.0% 0.1 N NaOH may be used, such as, e.g., 1.0%, 2.0%, 3.0%, 4.0%, 5.0%, 6.0%, 7.0%, 8.0%, 9.0% or 10.0% 0.1 N NaOH. Hence in another embodiment, the composition is obtained by combining about 1.15% to 1.8% (w/w) testosterone; about 0.6% to about 1.2% (w/w) isopropyl myristate; about 60% to about 80% (w/w) ethanol; about 0.6% to about 1.4% (w/w) carbomer; from about 6.5% to about 7.5% (w/w) 0.1N NaOH and additional water.

In yet another embodiment, the pharmaceutical composition includes testosterone in a hydroalcoholic gel. The concentration of testosterone in the gel can be varied. For example, the testosterone may be present in a concentration of about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%, about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, or about 10% weight to weight of the composition. The enhancer in this embodiment includes isopropyl myristate, which may be present in a concentration of about 0.5%, about 0.65%, about 0.75%, about 0.85%, about 0.95%, about 1%, about 2%, about 3%, about 4%, or about 5% weight to weight of the composition. The pharmaceutical composition also includes a C1-C4 alcohol present in a concentration of about 70%, about 71%, about 71.4%, about 71.8%, about 72%, about 72.3%, about 72.5%, about 72.7%, about 73%, about 73.5%, about 74%, about 74.5%, about 75% or about 75% weight to weight of the composition. Further, the pharmaceutical composition includes polyacrylic acid and/or carboxymethylcellulose as the gelling agent. In one embodiment, the gelling agent is polyacrylic acid present in a concentration of about 1% weight to weight of the composition.

One such testosterone gel has only recently been made available in the United States under the trademark AndroGel® by Unimed Pharmaceuticals, Inc., Marietta, Ga., the assignee of this application. In one embodiment, the gel is comprised of the following substances in approximate amounts:

TABLE 4
Composition of AndroGel ®

| | AMOUNT (w/w) | |
|---|---|---|
| SUBSTANCE | PER 100 g OF GEL | |
| Testosterone | 1.0 | g |
| Carbopol 980 | 0.90 | g |
| Isopropyl myristate | 0.50 | g |
| 0.1 N NaOH | 4.72 | g |
| Ethanol (96% v/v) | 71.4 | g* |
| Purified water (qs) | to 100 | g |

*Corresponding to 67 g of ethanol

One skilled in the art will appreciate that the constituents of this formulation may be varied in amounts yet continue to be within the spirit and scope of the present invention. For example, the composition may contain about 1% (w/w) Testosterone, about 0.9% (w/w) Carbopol 980, about 0.5% (w/w) Isopropyl myristate, about 4.72% (w/w) 0.1 N NaOH, about 71.4% (v/v) Ethanol (about 96% pure), and purified water up to 100%. In various instances, it may be preferable to utilize higher testosterone concentrations. Hence, in another example, the composition may contain from about 1.15% to about 1.8% (w/w) Testosterone, from about 0.6% to about 1.4% (w/w) Carbopol 980, from about 0.6% to about 1.2% (w/w) Isopropyl myristate, from about 6.5% (w/w) to about 7.5% 0.1 N NaOH, from about 60% to about 80% (v/v) Ethanol (about 96% pure), and purified water up to 100%. In another example, the composition may contain about 0.1 to about 10.0 g of testosterone, about 0.1 to about 5.0 g CARBOPOL, about 0.1 to about 5.0 g isopropyl myristate, and about 30.0 to about 98.0 g ethanol.

In still another embodiment, the composition comprises testosterone in an amount greater than 0.01%, a penetration enhancing agent in an amount greater than about 0.1%, a thickening agent in an amount greater than about 0.1%, and a lower alcohol in an amount greater than about 30% w/w of the composition.

The gel is rubbed or placed onto an area of skin of the subject and allowed to dry. The gel dries rapidly, i.e., within about 30 seconds to about 3 minutes after application. Illustratively, the gel is rubbed onto an area of skin, for example, on the upper outer thigh and/or hip once daily. Following application the subject washes his or her hands. Application of the gel results in an increased testosterone level having a desirable pharmacokinetic profile and is effective to treat or prevent hypogonadism and/or low testosterone, or the symptoms associated with, or related to hypogonadism and/or low testosterone in the subject. The composition is thus useful for treating a number of conditions or diseases in both adolescents under 18 years of age and adults 18 years of age and older.

In one embodiment, the present invention employs a packet having a polyethylene liner compatible with the components of a testosterone gel, as described below. The packet may hold a unit dose or multiple dose.

In another embodiment, the methods and compositions employ a composition that is dispensed from a rigid multi-dose container (for example, with a hand pump) having a larger foil packet, for example, of the composition inside the container. Such larger packets can also comprise a polyethylene liner as above. In one embodiment, the multi-dose container comprises an airless pump that comprises a polyethylene lined foil pouch within a canister with a hand pump inserted. In one embodiment, the polyethylene lined foil pouch comprises 44 g or 88 g of product. In one embodiment, the pump is capable of dispensing a total amount of about 75 g of gel. In one embodiment, the pump is primed before use, such as, e.g., by fully depressing the pump three times and discarding the gel. In one embodiment, the pump contains enough product to allow for priming and a set number of precise doses. In one embodiment, each full pump depression delivers 1.25 g of testosterone gel. In this embodiment, a 3.75 g dose of gel would require 3 pump depressions. A 5 g dose of gel would require 4 pump depressions. A 7.5 g dose of gel would require 6 pump depressions. A 10 g dose of gel would require 8 depressions, and so on. Of course, each pump depression can deliver any amount of testosterone gel suitable for delivering the desired dose. Indeed, in another embodiment, each full pump depression delivers 0.5 g of testosterone gel. In this embodiment, a 5 g dose of gel would require 10 pump depressions, and so on. The pouch size, amount dispensed and the delivery volume per depression are not limited to these embodiments and may be changed or adjusted to meet the needs of the patient population.

It has been shown, and is discussed in U.S. Pat. No. 6,503,894, U.S. Published Patent Applications 2002/0183296, 2003/0022877, 2003/0050292, 2003/0139384, 2003/0232072, 2004/0002482, 2004/0092494, and U.S. patent application Ser. Nos. 09/703,753, 10/787,071, 10/825,540, 10/828,678, 10/829,618, 10/867,435, 10/924,421, and 10/925,421, herein incorporated by reference in their entirety, that transdermal application of testosterone using AndroGel® to hypogonadal men results in improved testosterone levels, mood, libido and sexual performance. As disclosed herein, it has now been discovered that AndroGel® may also be used for the treatment of pediatric hypogonadism.

The methods and compositions of the present invention provide enhanced treatment options for treating, preventing, reversing, halting or slowing the progression of hypogonadism or another low-testosterone-associated disorder in a subject, for example, an adolescent male between 9 and 17 years of age (inclusive), as compared to those currently available.

In one embodiment, the pharmaceutical composition of the present invention is administered once, twice, or three times a day, or as many times necessary to achieve the desired therapeutic effect. In another embodiment the composition of the present invention is administered once, twice, or three times a day on alternate days. In another embodiment the composition of the present invention is administered once, twice, or three times a day on a weekly, biweekly, or monthly basis.

In one embodiment, a therapeutically effective dose is between about 0.5 g and under about 5.0 g, preferably between about 0.5 g and 2.5 g.

The composition is capable of releasing the steroid after applying the composition to the skin at a rate and duration that delivers in one embodiment of the present invention at least about 10 μg per day of the steroid to the blood serum of the subject.

In another embodiment of the present invention, the composition is capable of releasing the testosterone after applying the composition to the skin of a subject at a rate and duration that achieves a circulating serum concentration of testosterone greater than about 100 ng/dL serum.

In another embodiment of the present invention, the composition is capable of releasing the testosterone after applying the composition to the skin of a subject at a rate and duration that achieves a circulating serum concentration of total testosterone greater than about 100 ng/dL serum during a time period beginning about 0.5 hours after administration and ending about 24 hours after administration.

In another embodiment of the present invention, after administration of the composition, an obtained Cₘₐₓ is between about 100 and 1000 ng/dL.

In another embodiment of the present invention, the composition is provided to a subject for daily administration in about a 0.5 g to about a 2.5 g dose, such as, e.g., about 0.5 g, or about 1.5 g, or about 2.5 g. Any other suitable dose may be also be administered.

In yet another embodiment of the present invention, the subject in need of treatment has a serum testosterone level before the first application (pretreatment) of the composition of the present invention of less than about 100 ng/dL. In another embodiment of the present invention, the subject in need of treatment has a serum testosterone level before the first application (pretreatment) of the composition of the present invention of less than the normal range of an adolescent male in Tanner Stage II, i.e., less than between about 5 and about 70 ng/dL, as shown in Table 1.

In another embodiment of the present invention, where after at least about 30 days of daily administration of the composition of the present invention the serum testosterone concentration in a subject is at least about 100 ng/dL to about 1000 ng/dL, such as, for example, about 100 ng/dL to about 500 ng/dL, about 200 ng/dL to about 300 ng/dL, about 200 ng/dL to about 400 ng/dL, or about 200 ng/dL to about 500 ng/dL.

In still another embodiment of the present invention, where after daily administration of the composition of the present invention the total testosterone concentration in a subject is greater than about 100 ng/dL. In one embodiment, the total serum testosterone concentration in the subject is greater than about 200 ng/dL, about 300 ng/dL, about 400 ng/dL or about 500 ng/dL. In one embodiment, the total testosterone concentration is measured after 24 hours of administration. In one embodiment, the total testosterone concentration is measured after more than 2 days of daily administration, such as, for example, after 10 days, 14 days, 20 days, or 30 days.

In another embodiment of the methods, kits, combinations, and compositions of the present invention, the composition of the present invention is administered once, twice, or three times daily to a subject for at least about 4 days. In one embodiment, the composition is administered once a day.

The present invention is further illustrated by the following example, which should not be construed as limiting in any way. The contents of all cited references throughout this application are hereby expressly incorporated by reference. The practice of the present invention will employ, unless otherwise indicated, conventional techniques of pharmacology and pharmaceutics, which are within the skill of the art.

### EXAMPLES

### Example 1

### Pharmacokinetic Evaluation of Testosterone gel (1%) in Prepubertal Males of Adolescent Age

### Objectives

To evaluate the steady-state serum testosterone concentrations, the pharmacokinetic (PK) characteristics, and the safety and tolerability of testosterone gel 1% in prepubertal males of adolescent age with insufficient testosterone production. The primary evaluation of PK characteristics was based on steady-state PK parameters determined from serum total testosterone concentrations.

### Methods

Formulations: AndroGel®, 1% testosterone, was prepared and supplied by Solvay Pharmaceuticals, Inc.

Design: A multi-center, open-label, escalating-dose study conducted in up to 18 prepubertal boys of adolescent age. There was one treatment group and three treatment periods (Treatment Period 1, 2, and 3) during which subjects applied one of three escalating doses of testosterone gel 1% (0.5 g, 1.5 g, and 2.5 g containing 5 mg, 15 mg, and 25 mg of testosterone respectively) for 4 consecutive days. Each treatment period was separated by a washout period of up to 14 days.

A schematic of the study design is displayed in Table 5.

TABLE 5
Study Scheme
Pharmacokinetic Evaluation Phase

| | | Treatment Period 1 | | Treatment Period 2 | | Treatment Period 3 |
|---|---|---|---|---|---|---|
| | | 0.5 g of | | 1.5 g of | | 2.5 g of |
| | | Testosterone Gel 1% | Washout | Testosterone Gel 1% | Washout | Testosterone Gel 1% |
| Screening | Baseline | Visit 1 | Period 1 | Visit 2 | Period 2 | Visit 3 |
| Days − 14 to −1 | Day 0 | 4 days | no more | 4 days | no more | 4 days |
| | | | than 14 | | than 14 | |
| | | | days | | days | |
| → | → | → | | → | | → |

Treatments Administered: Three different doses of testosterone gel 1% were utilized in this study (0.5 g, 1.5 g, and 2.5 g), and administered topically during three treatment periods as indicated in Table 6. Testosterone gel 1% was supplied in multi-dose bottles with attached pumps calibrated to dispense 0.5 g of testosterone gel 1% as shown in Table 6.

TABLE 6
Determination of Dose of Testosterone-Gel
Dose of Testosterone-Gel (1%)

| Treatment Period 1: | Treatment Period 2: | Treatment Period 3: |
|---|---|---|
| 0.5 g (5 mg of | 1.5 g (15 mg of | 2.5 g (25 mg of |
| testosterone) | testosterone) | testosterone) |
| 1 metered-dose | 3 metered-dose | 5 metered-dose |
| actuation | actuations | actuations |

Each subject received a single dose of testosterone gel 1% over each 4 day period, with a 14 day washout period between treatments. Study drug was applied topically once daily in the morning. The following table lists the ingredients combined to yield the study formulation used.

TABLE 7
Ingredients Combined to Yield Study Formulation (% w/w)

| | | Amount (w/w) | |
|---|---|---|---|
| Component | Function | per 100 g | |
| Testosterone | Active pharmaceutical | 1.0 | g |
| | ingredient | | |
| Alcohol (95% v/v)* | Absorption enhancer | 71.4 | g |
| Isopropyl myristate | Absorption enhancer | 0.50 | g |
| Carbopol 980 | Thickening agent | 0.90 | g |
| | precursor | | |
| 0.1 N Sodium hydroxide | Neutralizer | 4.72 | g |
| Purified water (qs) | Solvent | to 100 | g |

*Equivalent to about 68.1% of absolute alcohol in the formulation.

Main Inclusion Criteria:

(a) Subject's parent or legal guardian have signed an informed consent and subjects have signed an assent according to local laws;

(b) Males 13-17 years of age, inclusive, with primary or secondary hypogonadism or CDGP;

(c) For testosterone naïve subjects, prepubertal maturation status, as indicated by: (i) Testis volume of ≦3 mL and (ii) Testosterone concentration of ≦50 ng/dL;

(d) Bone age of at least 10.5 years; and

(e) Hemoglobin of at least 12 g/dL and hematocrit of at least 36%.

Subjects: A total of seventeen (17) prepubertal boys of adolescent age were enrolled and provided serum concentration data for evaluation at the 0.5 g/day and 1.5 g/day dose levels. Four of 17 subjects did not complete the 2.5 g/day dose level due to achieving serum testosterone values greater than 200 ng/dL. Therefore thirteen (13) subjects provided serum concentration data at the 2.5 g/day dose level. Subjects who were discontinued due to exceeding a serum testosterone level of 200 ng/dL were considered study completers due to the protocol defined upper limit of 200 ng/dL. Of the seventeen (17) subjects enrolled, thirteen (13) subjects were diagnosed with primary or secondary hypogonadism and four (4) subjects were diagnosed with CDGP.

Subject Demographics: Table 8 provides the summary statistics of demographic and Baseline characteristics for all subjects.

TABLE 8
Demographic Characteristics of All Subjects

| | | Subject Population | | | | | |
|---|---|---|---|---|---|---|---|
| | Statistic | CDGP | | Hypogonadal | | All Subjects | |
| Parameter | n | 4 | | 13 | | 17 | |
| Age | Mean (SD) | 14.5 | (1.29) | 14.8 | (1.57) | 14.8 | (1.48) |
| Race | | | | | | | |
| White | n (%) | 4 | (100) | 9 | (69.2) | 13 | (76.5) |
| Black or African American | n (%) | 0 | | 1 | (7.7) | 1 | (5.9) |
| American Indian or Alaskan Native | n (%) | 0 | | 0 | | 0 | |
| Asian | n (%) | 0 | | 0 | | 0 | |
| Native Hawaiian or Other Pacific | n (%) | 0 | | 0 | | 0 | |
| Islander | | | | | | | |
| Two or More Races | n (%) | 0 | | 0 | | 0 | |
| Unknown | n (%) | 0 | | 3 | (23.1) | 3 | (17.6) |
| Ethnicity | | | | | | | |
| Hispanic or Latino | n (%) | 0 | | 3 | (23.1) | 3 | (17.6) |
| Not Hispanic or Latino | n (%) | 4 | (100) | 10 | (76.9) | 14 | (82.4) |
| Height (cm) | Mean (SD) | 162.5 | (15.24) | 166.1 | (12.18) | 165.3 | (12.54) |
| Weight (kg) | Mean (SD) | 55.5 | (21.78) | 61.8 | (12.71) | 60.3 | (14.75) |

Note:
Percentages are based on the number of subjects who received study medication.
Note:
Three subjects are reported with race as missing, however, the CRF page for demographic data was revised during the study: from recording race (including the category of Hispanic) to recording both race and ethnicity. These subjects were reported as of Hispanic race and this was subsequently captured as Hispanic or Latino ethnicity and race was recorded as missing.

### Procedures and Assessments

Dose Administration: Three escalating doses of testosterone gel 1% (0.5 g, 1.5 g, and 2.5 g containing 5 mg, 15 mg, and 25 mg of testosterone respectively) were applied once daily in the morning for four consecutive days with a washout period between each dose. Subjects were instructed to wash the application site with soap and water 8-10 hours after each dose.

Washout Period was defined as the period between the day after the last application of study medication in the last treatment period visit and the day before the first application in the next treatment visit (inclusive). Each treatment period was separated by a washout period of up to 14 days.

Pharmacokinetic Sampling: Blood samples for pharmacokinetic (PK) measurement of serum concentrations of total, bioavailable, and free testosterone and total DHT were collected five minutes prior to testosterone gel 1% application (predose), and at 1, 2, 4, 8, 12, and 24 hours after application on Day 4 of Treatment Periods 1, 2, and 3. Samples were also collected at the same nominal time points on Day 0, which was the day before the first dose, based on the “projected dosing time” during the treatment periods. An optional blood sample may have been collected between 2 and 12 hours after testosterone gel 1% application on Day 1 of Treatment Periods 1, 2, and 3.

Bioanalytical Method: Measurements of total, free, and bioavailable testosterone, as well as total DHT, E2, FSH, LH, and SHBG were performed at Esoterix Laboratory Services, 4301 Lost Hills Road, Calabasas Hills, Calif. 91301.

### Criteria for Evaluation

Safety: Vital signs, ECG, physical examination, clinical laboratory determinations (including PSA measurement), DRE and IPSS, safety testosterone and hematocrit measurements.

Pharmacokinetic Analyses: Serum concentrations (i.e., observed concentrations) for total, free, and bioavailable testosterone, and total DHT were summarized descriptively (n, mean, SD, CV %, minimum, maximum, median and geometric mean) for each serial sampling time at Baseline (Day 0) and for each treatment period (on Day 4 of Treatment Periods 1, 2 and 3). Additionally, Baseline-adjusted concentrations for each treatment period for total, free, and bioavailable testosterone, and total DHT were also summarized descriptively. The summary data was presented for all subjects (17 subjects), subjects with hypogonadism (13 subjects) and subjects with CDGP (4 subjects) separately. Pharmacokinetic parameters included the following:

(a) AUC_{0-24,ss}: area under the curve from 0 to 24 hours, determined using the linear trapezoidal rule; a minimum of four data points were required for the calculation of AUC; otherwise AUC was defined as missing;

(b) C_{max,ss}: maximum observed concentration over 24-hour dosing interval;

(c) t_{max,ss}: time at which Cₘₐₓ occurred;

(d) C_{min,ss}: lowest concentration observed during the 24-hour dosing interval;

(e) t_{min,ss}: time at which Cₘᵢₙ occurred;

(f) C_{avg,ss}: the time-averaged concentration over the dosing interval, determined by AUC₀₋₂₈/24;

(g) Fluctuation Index: the extent of variation in the serum concentration over the course of a single day, calculated as (Cₘₐₓ−Cₘᵢₙ)/C_{avg};

Statistical methods: Summary statistics for selected data collected during this study are presented to give a general description of the subjects studied and an overview of the PK and safety results. Categorical variables are summarized by presenting the number and percentage of subjects in each category. Continuous variables are summarized using n, mean, SD, median, minimum value, and maximum value.

### Screening Testosterone Baseline Values

At screening, all subjects naïve to testosterone had testosterone concentrations ≦50 ng/dL, confirming their hypogonadal status prior to exposure to study drug. Approximately two-thirds of all subjects were naïve to androgen therapy prior to entering the study (11 subjects, 64.7%). Table 9 provides the screening baseline serum hormone concentrations for all subjects.

TABLE 9
Baseline Characteristics for All Subjects

Parameter

| Was the Subject Naive to Androgen Therapy | | Subject Population | | | | | |
|---|---|---|---|---|---|---|---|
| Prior to Entering Study | Statistic | CDGP | | Hypogonadal | | All Subjects | |
| Yes | n (%) | 3 | (75.0%) | 8 | (61.5%) | 11 | (64.7%) |
| No | n (%) | 1 | (25.0%) | 5 | (38.5%) | 6 | (35.3%) |
| Serum Hormone Concentrations | n | 4 | | 13 | | 17 | |
| Total Testosterone (ng/dL) | Mean (SD) | 97.3 | (97.80) | 62.3 | (118.90) | 70.5 | (112.38) |
| | Median | 85.0 | | 17.0 | | 19.0 | |
| | Range | 3.0, 216.0 | | 3.0, 421.0 | | 3.0, 421.0 | |
| Free Testosterone (ng/dL) | Mean (SD) | 6.4 | (6.17) | 7.8 | (12.91) | 7.5 | (11.51) |
| | Median | 5.5 | | 2.5 | | 2.5 | |
| | Range | 0.5, 14.0 | | 0.7, 38.0 | | 0.5, 38.0 | |
| Total DHT (ng/dL) | Mean (SD) | 14.9 | (13.19) | 8.1 | (9.71) | 9.7 | (10.59) |

| | | | |
|---|---|---|---|
| Median | 12.8 | 4.9 | 5.4 |
| Range | 2.0, 32.0 | 2.0, 36.0 | 2.0, 36.0 |

Note:
Percentages are based on the number of subjects who received study medication.

The mean serum total concentration was 70.5 ng/dL, but the mean appears to be skewed as the median serum total concentration was 19.0 ng/dL.

### Pharmacokinetic and Pharmacodynamic Results

Out of the 17 subjects enrolled in the study, 13 subjects completed all the three treatment periods (0.5, 1.5 and 2.5 g treatment with testosterone 1% gel). Four subjects did not complete the last treatment period as their serum testosterone exceeding a level of >200 ng/dL.

### Testosterone Concentration-Time Data

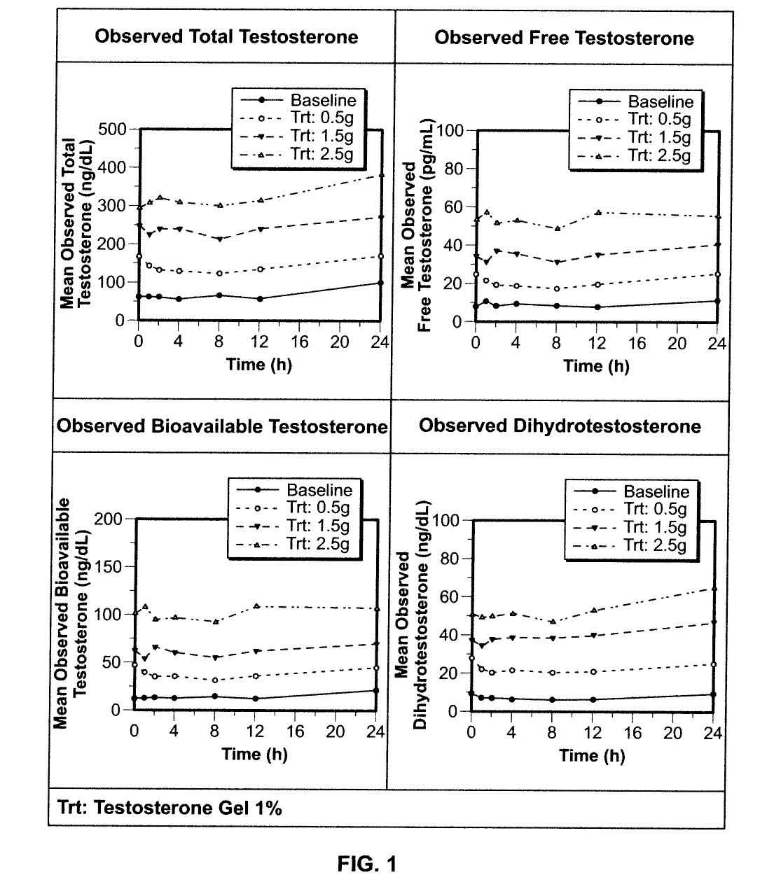

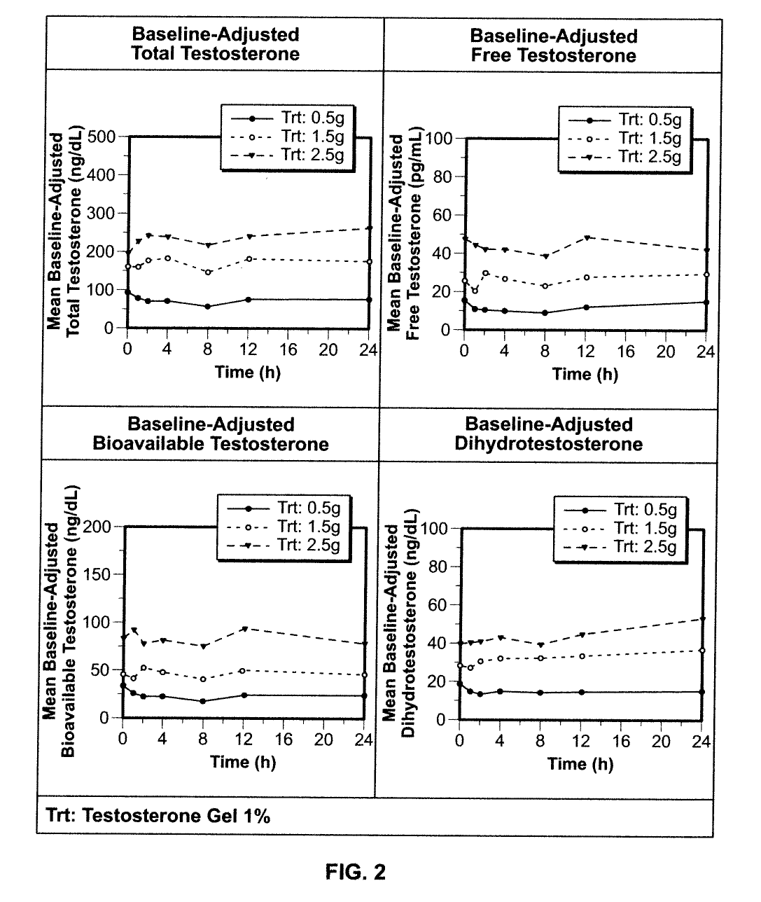

FIG. 1 shows the observed mean concentration profiles for total, free, bioavailable testosterone and total DHT for all treatment groups. FIG. 2 shows the Baseline-adjusted mean concentration profiles for total, free, bioavailable testosterone and total DHT for all treatment groups.

Referring to FIGS. 1 and 2, a dose-related increase in total testosterone concentrations (observed) over the entire concentration-time profile was observed with each increase in dose (0.5, 1.5, and 2.5 g doses of testosterone gel 1%, containing 5, 15, and 25 mg of testosterone, respectively). At all testosterone gel dose levels, total testosterone concentrations were notably increased compared to Baseline. Mean serum concentration-time profiles were quite flat, indicating that testosterone concentrations were maintained at fairly constant levels throughout the day. Concentrations at 24 hours postdose were comparable to predose within each dose group except the 2.5 g dose group, suggesting that total testosterone concentrations were representative of steady-state conditions on Day 4. The mean concentration data at 24 hours postdose for the 2.5 g treatment group was higher than the predose and this could be due to the contribution of an anomalous total testosterone concentration from Subject 201/2004 that was 2 to 5-fold higher than the rest of the subjects in the same treatment group. Profiles of observed concentrations for free and bioavailable testosterone and total DHT approximately paralleled results observed for total testosterone. The ‘Baseline-adjusted’ concentration-time profiles for all treatment groups and for all analytes followed a similar pattern.

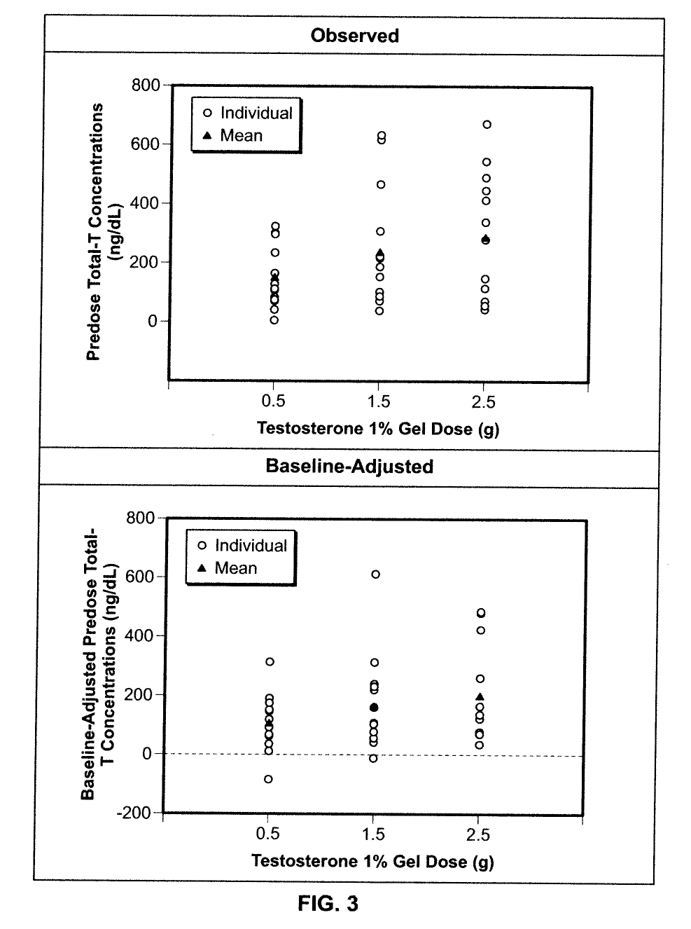

FIG. 3 shows the predose levels of observed and Baseline adjusted total testosterone for all treatment periods before treatment with 0.5 g, 1.5 g, and 2.5 g of testosterone gel 1%. In general, the mean predose concentrations increased with increase in dose.

### Pharmacokinetic Parameters

Table 10 summarizes the observed PK parameters for total testosterone, free testosterone, and total DHT after treatment.

TABLE 10
Summary of Observed Pharmacokinetic Parameters in All Subjects After Treatment

| | | Arithmetic Mean (SD) | | | | | |
|---|---|---|---|---|---|---|---|
| Analyte | Parameter | 0.5 g (n = 17) | | 1.5 g (n = 17) | | 2.5 g (n = 13) | |
| Total-T | C_{max, ss} (ng/dL) | 211.3 | (147) | 361.0 | (217.8) | 492.8 | (291.7) |
| | C_{avg, ss} (ng/dL) | 140.5 | (111.7) | 241.8 | (133.70) | 326.0 | (188.0) |
| | t_{max, ss} (h)^{[a]} | 2.00 | (0.0-24.03) | 4.00 | (0.00-24.25) | 12.08 | (0.0-24.0) |
| | AUC_{0-24, ss} (ng*h/dL) | 3372 | (2683) | 5808 | (3220) | 7853 | (4511) |
| Free-T | C_{max, ss} (pg/mL) | 31.80 | (22.47) | 53.94 | (29.47) | 86.31 | (62.44) |
| | C_{avg, ss} (pg/mL) | 19.67 | (15.01) | 34.93 | (15.68) | 53.73 | (42.76) |
| | t_{max, ss} (h)^{[a]} | 2.00 | (0.0-24.03) | 4.00 | (0.00-24.25) | 12.00 | (0.0-24.0) |
| | AUC_{0-24, ss} (pg*h/mL) | 472.4 | (361.0) | 839.2 | (376.8) | 1294 | (1027) |
| Total DHT | C_{max, ss} (ng/dL) | 31.71 | (17.15) | 53.29 | (38.78) | 76.31 | (43.58) |
| | C_{avg, ss} (ng/dL) | 21.94 | (12.20) | 40.50 | (26.34) | 54.03 | (33.87) |
| | t_{max, ss} (h)^{[a]} | 8.00 | (0.0-25.00) | 12.00 | (0.00-24.25) | 12.00 | (0.0-24.0) |
| | AUC_{0-24, ss} (ng*h/dL) | 527.2 | (293.3) | 974.4 | (637.8) | 1301 | (813) |
| Bioavailable T | C_{max, ss} (ng/dL) | 59.70 | (44.82) | 100.4 | (63.0) | 163.3 | (125.1) |
| | C_{avg, ss} (ng/dL) | 37.11 | (29.23) | 61.70 | (30.84) | 102.1 | (89.6) |
| | t_{max, ss} (h)^{[a]} | 2.00 | (0.0-24.03) | 4.00 | (0.0-24.25) | 12.0 | (0.0-24.0) |
| | AUC_{0-24, ss} (ng*h/dL) | 889.8 | (700.3) | 1428 | (742) | 2462 | (2158) |

^{[a]}The estimate is the median value and range for the PK parameter.
0.5 g = 500 mg of testosterone gel 1%
1.5 g = 1500 mg of testosterone gel 1%
2.5 g = 2500 mg of testosterone gel 1%
Total T = total testosterone
Free T = free testosterone
Bioavailable T = bioavailable testosterone
DHT = dihydrotestosterone

Referring to Table 10, the observed median tₘₐₓ for total testosterone ranged from 2 to 12 hours across the 3 treatment periods. A dose-related increase in mean exposure (mean AUC_{0-24,ss}, C_{max,ss}, and C_{avg,ss}) to total testosterone was observed with increasing dose, though this increase was less than dose-proportional. The parameters AUC_{0-24,ss}, C_{max,ss}, and C_{avg,ss} showed a 2.3-fold increase over a 5-fold increase in dose (5 mg to 25 mg) of testosterone, respectively. Similar results were observed for free and bioavailable testosterone.

For total DHT, the observed median tₘₐₓ ranged from 8 to 12 hours across the three treatment periods. The parameters AUC_{0-24,ss}, C_{max,ss}, and C_{avg,ss} showed a 2.5-fold increase over a 5-fold increase in dose (5 mg to 25 mg) of testosterone, respectively.

Table 11 below provides the Baseline-adjusted PK parameters for total, free and bioavailable testosterone and total DHT. The Baseline-adjusted median tₘₐₓ for total testosterone ranged from 2 to 8.08 hrs across the 3 treatment periods. For the total, free and bioavailable testosterone and total DHT baseline-adjusted parameters, the trends were similar to those of ‘observed’ PK parameters.

TABLE 11
Summary of Baseline-Adjusted Pharmacokinetic Parameters in All Subjects

| | | Arithmetic Mean (SD) | | | | | |
|---|---|---|---|---|---|---|---|
| Analyte | Parameter | 0.5 g (n = 17) | | 1.5 g (n = 17) | | 2.5 g (n = 13) | |
| Total-T | C_{max, ss} (ng/dL) | 137.3 | (66.6) | 288.5 | (177.8) | 387.5 | (311.3) |
| | C_{avg, ss} (ng/dL) | 67.85 | (36.61) | 166.9 | (98.5) | 227.4 | (189.1) |
| | t_{max, ss} (h)^{[a]} | 2.0 | (0.0-24.03) | 4.00 | (0.00-24.25) | 8.08 | (0.00-24.00) |
| | AUC_{0-24, ss} (ng*h/dL) | 1634 | (883) | 4014 | (2382) | 5482 | (4539) |
| Free-T | C_{max, ss} (pg/mL) | 22.72 | (13.17) | 44.47 | (28.63) | 75.02 | (64.14) |
| | C_{avg, ss} (pg/mL) | 10.94 | (6.69) | 25.70 | (16.64) | 41.41 | (42.24) |
| | t_{max, ss} (h)^{[a]} | 12.0 | (0.0-24.03) | 4.00 | (0.00-24.25) | 12.03 | (0.00-24.00) |
| | AUC_{0-24, ss} (pg*h/mL) | 263.1 | (160.4) | 617.8 | (400.5) | 999.5 | (1019.2) |
| Total DHT | C_{max, ss} (ng/dL) | 23.86 | (12.60) | 45.01 | (36.36) | 67.15 | (43.87) |
| | C_{avg, ss} (ng/dL) | 14.77 | (8.50) | 33.36 | (24.76) | 45.28 | (32.69) |
| | t_{max, ss} (h)^{[a]} | 7.98 | (0.00-24.00) | 12.00 | (0.00-24.25) | 12.00 | (0.00-24.00) |
| | AUC_{0-24, ss} (ng*h/dL) | 355.3 | (204.8) | 802.5 | (599.8) | 1090 | (785) |
| Bioavailable T | C_{max, ss} (ng/dL) | 43.28 | (27.12) | 81.65 | (62.07) | 141.5 | (125.6) |
| | C_{avg, ss} (ng/dL) | 21.20 | (13.72) | 44.92 | (32.39) | 79.26 | (86.86) |
| | t_{max, ss} (h)^{[a]} | 4.0 | (0.0-24.03) | 4.0 | (0.0-24.25) | 4.0 | (0.0-24.0) |
| | AUC_{0-24, ss} (ng*h/dL) | 509.4 | (328.5) | 1079 | (779) | 1911 | (2091) |

^{[a]}The estimate is the median value and range for the PK parameter.
0.5 g = 500 mg of testosterone gel 1%
1.5 g = 1500 mg of testosterone gel 1%
2.5 g = 2500 mg of testosterone gel 1%
Total T = total testosterone
Free T = free testosterone
Bioavailable T = bioavailable testosterone
Total DHT = dihydrotestosterone

### CONCLUSIONS

Pharmacokinetics of total and free testosterone and total DHT was characterized in pediatric population of hypogonadal young males after the topical application of 0.5 g, 1.5 g and 2.5 g testosterone gel 1%.

Steady-state concentration-time profiles were relatively flat for all analytes, indicating that concentrations of these analytes remained at fairly stable levels throughout the dosing interval.

A dose-related increase in exposure (AUC_{0-24,ss}, C_{max,ss}, C_{avg,ss}) was observed for total and free testosterone with increasing doses of testosterone in comparison to Baseline concentrations. Although the increase was not dose-proportional, there was no indication of departure from linear pharmacokinetics.

Testosterone gel 1% appears to be safe and well-tolerated in this pediatric subject population as there were no deaths or other significant adverse events during this study. There were also no clinically meaningful changes from Baseline to Final Visit during the Pharmacokinetic Evaluation Phase for any hematology, blood chemistry, urinalysis, or lipid parameters.

All references, including publications, patent applications, and patents, cited herein are hereby incorporated by reference to the same extent as if each reference were individually and specifically indicated to be incorporated by reference and were set forth in its entirety herein.

The uses of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the particular subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it there individually recited herein.

Use of the phrase ‘the invention’ or ‘the present invention’ is not meant to limit the claims in any manner and no conclusion should be drawn that any description or argument associated with a particular use of the phrase ‘the invention’ or ‘the present invention’ applies to each and every claim. The use of the phrase ‘the invention’ or ‘the present invention’ has been used solely for linguistic or grammatical convenience and not to effect a limitation of any nature on any of the claims.

Alternative embodiments of the claimed invention are described herein, including the best mode known to the inventors for carrying out the claimed invention. Of these, variations of the disclosed embodiments will become apparent to those of ordinary skill in the art upon reading the foregoing disclosure. The inventors expect skilled artisans to employ such variations as appropriate, and the inventors intend for the claimed invention to be practiced otherwise than as specifically described herein. Accordingly, the claimed invention includes all modifications and equivalents of the subject matter recited in the claims appended hereto as permitted by applicable law. Moreover, any combination of the above-described elements in all possible variations thereof is encompassed by the claimed invention unless otherwise indicated herein or otherwise clearly contradicted by context.

It is to be understood that any ranges, ratios and ranges of ratios that can be formed by, or derived from, any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art most closely related to a particular range, ratio or range of ratios will appreciate that such values are unambiguously derivable from the data presented herein.

The use of the terms “a” and “an” and “the” and similar referents in the context of this disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope of the claims. No language in the specification should be construed as indicating any non-claimed element as essential to the practice of the claimed invention.

What is claimed is:

1. A method of treating hypogonadism in an adolescent boy comprising administering a topical pharmaceutical composition in the form of a hydroalcoholic gel containing testosterone to said adolescent boy.

2. The method of claim 1, further comprising determining that the adolescent boy has a testosterone level below the appropriate level for his age.

3. The method of claim 2, further comprising raising the serum testosterone level of said boy to a level lying within the eugonadal range for his age.

4. The method of claim 1, wherein the adolescent boy is between 9 and 17 years old, inclusive.

5. The method of claim 1, wherein the adolescent boy is between 15 and 17 years old, inclusive.

6. The method of claim 3, further comprising targeting a eugonadal serum testosterone level that lies in the range from 5 to 50 ng/dL total testosterone for the age from 10 to 11 years, in the range from 10 to 570 ng/dL total testosterone for the age from 12 to 14 years, and in the range from 220 to 800 ng/dL total testosterone for the age from 15 to 17 years.

7. The method of claim 1, wherein the testosterone is administered to the skin of said adolescent boy in a daily dose of between about 5 mg and 25 mg testosterone.

8. The method of claim 1, wherein the hydroalcoholic gel comprises:
a. about 0.01 to about 15% (w/w) testosterone;
b. about 0.01 to about 50% (w/w) penetration enhancing agent;
c. about 0.01 to about 50% (w/w) gelling agent;
d. about 30 to about 98% (w/w) of a lower alcohol; and
e. purified water up to 100% (w/w).

9. The method of claim 8, wherein the hydroalcoholic gel comprises:
a. about 0.9 to about 1.1% (w/w) testosterone;
b. about 0.85 to about 0.95% (w/w) Carbopol 980;
c. about 0.4 to about 0.6% (w/w) isopropyl myristate;
d. about 4.6 to about 4.9% (w/w) 0.1 N NaOH;
e. about 68 to about 73% (v/v) ethanol (96% pure); and
f. purified water up to 100%.

10. The method of claim 8, wherein the hydroalcoholic gel comprises:
a. about 1.15 to about 1.8% (w/w) testosterone;
b. about 0.6 to about 1.4% (w/w) Carbopol 980;
c. about 0.6 to about 1.2% (w/w) isopropyl myristate;
d. about 6.5 to about 7.5% (w/w) 0.1 N NaOH;
e. about 60 to about 80% (v/v) ethanol (96% pure); and
f. purified water up to 100%.

11. The method of claim 9, wherein the topical pharmaceutical composition in the form of a hydroalcoholic gel comprises: 1.0% (w/w) testosterone, 0.90 g Carbopol 980, 0.50 g isopropyl myristate, 4.72 g 0.1 N NaOH, 71.4 g 96% (v/v) ethanol, and purified water up to 100 g.

12. The method of claim 6, wherein the topical pharmaceutical composition in the form of a hydroalcoholic gel comprises: about 1.15 to about 1.80% (w/w) testosterone; about 0.6 to about 1.4 g Carbopol 980; about 0.6 to about 1.2 g isopropyl myristate; about 6.5 to about 7.5 g 0.1 N NaOH; about 60 to about 80 g 96% (v/v) ethanol; and purified water up to 100 g.

13. A method of treating delayed progression of an adolescent boy into puberty comprising administering a topical pharmaceutical composition in the form of a hydroalcoholic gel containing testosterone to said adolescent boy.

14. The method of claim 13, further comprising identifying an adolescent boy who has failed to progress into the Tanner Stage appropriate for his age and determining his serum testosterone level.

15. The method of claim 14, further comprising raising the serum testosterone level of said boy to a level lying within the range appropriate for the Tanner Stage of his age.

16. The method of claim 13, wherein the testosterone is administered to the skin of said adolescent boy in a daily dose of between about 5 mg and 25 mg testosterone.

17. The method of claim 13, wherein the hydroalcoholic gel comprises:
a. about 0.01 to about 15% (w/w) testosterone;
b. about 0.01 to about 50% (w/w) penetration enhancing agent;
c. about 0.01 to about 50% (w/w) gelling agent;
d. about 30 to about 98% (w/w) of a lower alcohol; and
e. purified water up to 100% (w/w).

18. The method of claim 17, wherein the hydroalcoholic gel comprises:
a. about 0.9 to about 1.1% (w/w) testosterone;
b. about 0.85 to about 0.95% (w/w) Carbopol 980;
c. about 0.4 to about 0.6% (w/w) isopropyl myristate;
d. about 4.6 to about 4.9% (w/w) 0.1 N NaOH;
e. about 68 to about 73% (v/v) ethanol (96% pure); and
f. purified water up to 100%.

19. The method of claim 17, wherein the hydroalcoholic gel comprises:
a. about 1.15 to about 1.8% (w/w) testosterone;
b. about 0.6 to about 1.4% (w/w) Carbopol 980;
c. about 0.6 to about 1.2% (w/w) isopropyl myristate;
d. about 6.5 to about 7.5% (w/w) 0.1 N NaOH;
e. about 60 to about 80% (v/v) ethanol (96% pure); and
f. purified water up to 100%.

20. The method of claim 18, wherein the hydroalcoholic gel comprises: 1.0% (w/w) testosterone, 0.90 g Carbopol 980, 0.50 g isopropyl myristate, 4.72 g 0.1 N NaOH, 71.4 g 96% (v/v) ethanol, and purified water up to 100 g.

21. The method of claim 15, wherein the hydroalcoholic gel comprises: about 1.15 to about 1.80% (w/w) testosterone; about 0.6 to about 1.4 g Carbopol 980; about 0.6 to about 1.2 g isopropyl myristate; about 6.5 to about 7.5 g 0.1 N NaOH; about 60 to about 80 g 96% (v/v) ethanol; and purified water up to 100 g.

22. A method of treating the failure of an adolescent boy to progress into puberty by age fourteen comprising administering a topical pharmaceutical composition in the form of a hydroalcoholic gel containing testosterone to said adolescent boy.

23. The method of claim 22, further comprising determining the serum testosterone level of said adolescent boy.

24. The method of claim 23, further comprising raising the serum testosterone of said boy to a level being sufficient to initiate his progression into adolescence.

25. The method of claim 22, wherein the adolescent boy is prepubertal.

26. The method of claim 22, wherein the testosterone is administered to the skin of said adolescent boy in a daily dose of between about 5 mg and 25 mg testosterone.

27. The method of claim 22, wherein the hydroalcoholic gel comprises:
a. about 0.01 to about 15% (w/w) testosterone;
b. about 0.01 to about 50% (w/w) penetration enhancing agent;
c. about 0.01 to about 50% (w/w) gelling agent;
d. about 30 to about 98% (w/w) of a lower alcohol; and
e. purified water up to 100% (w/w).

28. The method of claim 27, wherein the hydroalcoholic gel comprises:
a. about 0.9 to about 1.1% (w/w) testosterone;
b. about 0.85 to about 0.95% (w/w) Carbopol 980;
c. about 0.4 to about 0.6% (w/w) isopropyl myristate;
d. about 4.6 to about 4.9% (w/w) 0.1 N NaOH;
e. about 68 to about 73% (v/v) ethanol (96% pure); and
f. purified water up to 100%.

29. The method of claim 27, wherein the hydroalcoholic gel comprises:
a. about 1.15 to about 1.8% (w/w) testosterone;
b. about 0.6 to about 1.4% (w/w) Carbopol 980;
c. about 0.6 to about 1.2% (w/w) isopropyl myristate;
d. about 6.5 to about 7.5% (w/w) 0.1 N NaOH;
e. about 60 to about 80% (v/v) ethanol (96% pure); and
f. purified water up to 100%.

30. The method of claim 28, wherein the hydroalcoholic gel comprises: 1.0% (w/w) testosterone, 0.90 g Carbopol 980, 0.50 g isopropyl myristate, 4.72 g 0.1 N NaOH, 71.4 g 96% (v/v) ethanol, and purified water up to 100 g.

31. The method of claim 24, wherein the hydroalcoholic gel comprises: about 1.15 to about 1.80% (w/w) testosterone; about 0.6 to about 1.4 g Carbopol 980; about 0.6 to about 1.2 g isopropyl myristate; about 6.5 to about 7.5 g 0.1 N NaOH; about 60 to about 80 g 96% (v/v) ethanol; and purified water up to 100 g.

32. A pharmaceutical packet containing a hydroalcoholic gel comprising 5 mg, 15 mg, or 25 mg testosterone.

33. A multi-dose container containing a hydroalcoholic gel whereby a metered amount of gel can be dispensed per actuation (s) of a pump to deliver 5 mg, 15 mg or 25 mg testosterone.
